(19) United States
(12) Patent Application Publication
(10) Pub. No.: US 2018/0186765 A1
(43) Pub. Date: Jul. 5, 2018

(54) PROCESSES FOR THE PREPARATION OF PESTICIDAL COMPOUNDS

(71) Applicant: Dow AgroSciences LLC, Indianapolis, IN (US)

(72) Inventors: Qiang YANG, Indianapolis, IN (US); Yu ZHANG, Indianapolis, IN (US); Beth LORSBACH, Indianapolis, IN (US); Xiaoyong LI, Midland, MI (US); Gary ROTH, Midland, MI (US)

(21) Appl. No.: 15/853,089

(22) Filed: Dec. 22, 2017

Related U.S. Application Data
(60) Provisional application No. 62/440,178, filed on Dec. 29, 2016.

(51) Int. Cl. C07D 401/04; A01N 43/56

(52) U.S. Cl. CPC C07D 401/04; A01N 43/56

(57) ABSTRACT

This application relates to efficient and economical synthetic chemical processes for the preparation of pesticidal thioethers and pesticidal sulfoxides. Further, the present application relates to certain novel compounds necessary for their synthesis. It would be advantageous to produce pesticidal thioethers and pesticidal sulfoxides efficiently and in high yield from commercially available starting materials.

### CROSS-REFERENCE TO RELATED APPLICATIONS

This application claims priority under 35 U.S.C. § 119(e) to U.S. Provisional Application Ser. No. 62/440,178 filed Dec. 29, 2016, which is incorporated herein by this reference in its entirety.

### TECHNICAL FIELD OF THE DISCLOSURE

This application relates to efficient and economical synthetic chemical processes for the preparation of pesticidal thioethers and pesticidal sulfoxides. Further, the present application relates to certain novel compounds necessary for their synthesis. It would be advantageous to produce pesticidal thioethers and pesticidal sulfoxides efficiently and in high yield from commercially available starting materials.

### BACKGROUND

There are more than ten thousand species of pests that cause losses in agriculture. The world-wide agricultural losses amount to billions of U.S. dollars each year. Stored food pests eat and adulterate stored food. The world-wide stored food losses amount to billions of U.S. dollars each year, but more importantly, deprive people of needed food. Certain pests have developed resistance to pesticides in current use. Hundreds of pest species are resistant to one or more pesticides. The development of resistance to some of the older pesticides, such as DDT, the carbamates, and the organophosphates, is well known. But resistance has even developed to some of the newer pesticides. As a result, there is an acute need for new pesticides that has led to the development of new pesticides. Specifically, US 20130288893(A1) describes, inter alia, certain pesticidal thioethers and their use as pesticides. Such compounds are finding use in agriculture for the control of pests.

In U.S. Pat. No. 9,102,654, processes for preparing such pesticidal thioethers were described. In one embodiment, the intermediate 1d, described therein, was prepared according to the process shown in Scheme 1 below.

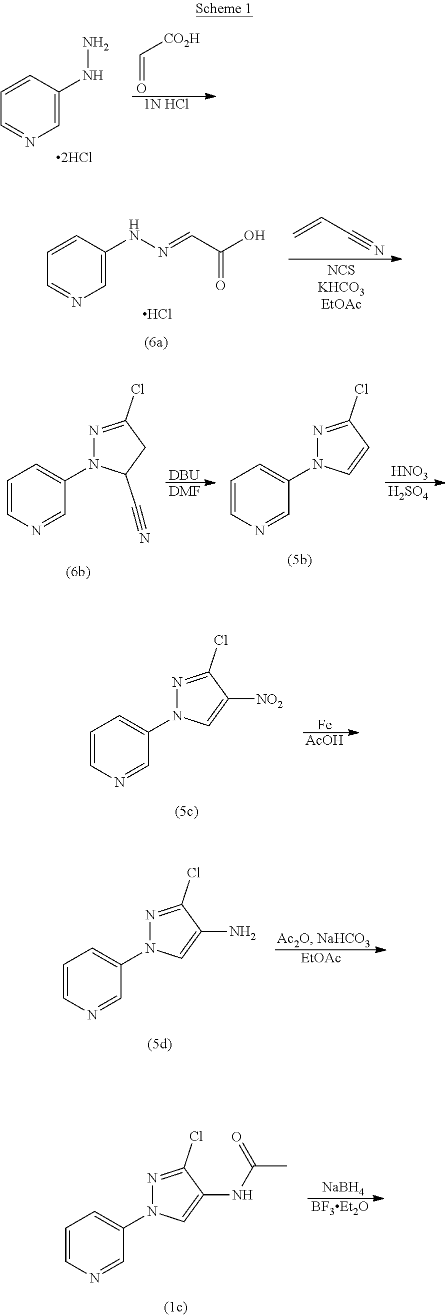

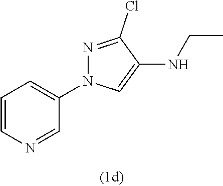

The process in Scheme 1 requires seven steps from commercially available starting material 3-hydrazinopyridine dihydrochloride to arrive at the compound of the formula 1d, by way of compound 5d, making the large scale manufacture of the target pesticidal thioethers described in U.S. Pat. No. 9,102,654 difficult.

Because there is a need for very large quantities of pesticides, particularly pesticidal thioethers of the type described in U.S. Pat. No. 9,102,654 and US Patent Publication 20130288893(A1), it would be highly advantageous to develop new processes to produce pesticidal thioethers efficiently and in high yield from economical commercially available starting materials.

### Definitions of the Disclosure

The following definitions apply to the terms as used throughout this specification, unless otherwise limited in specific instances.

As used herein, the term “alkyl” includes a chain of carbon atoms, which is optionally branched including but not limited to C₁-C₆, C₁-C₄, and C₁-C₃. Illustrative alkyl groups include, but are not limited to, methyl, ethyl, n-propyl, isopropyl, n-butyl, isobutyl, sec-butyl, tert-butyl, pentyl, 2-pentyl, 3-pentyl, and the like. Alkyl may be substituted or unsubstituted. It will be understood that “alkyl” may be combined with other groups, such as those provided above, to form a functionalized alkyl. By way of example, the combination of an “alkyl” group, as described herein, with a “cycloalkyl” group may be referred to as a “alkyl-cycloalkyl” group.

As used herein, the term “cycloalkyl” refers to an all-carbon cyclic ring, optionally containing one or more double bonds but the cycloalkyl does not contain a completely conjugated pi-electron system. It will be understood that in certain embodiments, cycloalkyl may be advantageously of limited size, such as C₃-C₆.

Cycloalkyl may be unsubstituted or substituted. Examples of cycloalkyl include cyclopropyl, cyclobutyl, and cyclohexyl.

As used herein, the term “aryl” refers to an all-carbon cyclic ring containing a completely conjugated pi-electron system. It will be understood that in certain embodiments, aryl may be advantageously of limited size, such as C₆-C₁₀. Aryl may be unsubstituted or substituted. Examples of aryl include phenyl and naphthyl.

As used herein, “halo” or “halogen” or “halide” may be used interchangeably and refers to fluorine (F), chlorine (Cl), bromine (Br) or iodine (I).

As used herein, “trihalomethyl” refers to a methyl group having three halo substituents, such as a trifluoromethyl group.

### DETAILED DESCRIPTION OF THE DISCLOSURE

The compounds and process of the present disclosure are described in detail below. The processes of the present disclosure can be described according to Scheme 2.

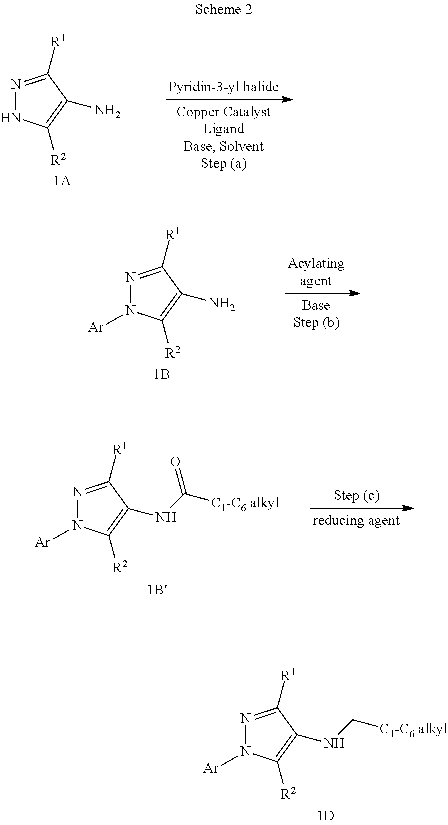

In Step (a) of Scheme 2, the pyrazole starting material 1A, wherein each of R¹ and R² is independently selected from the group consisting of H, F, Cl, Br, I, C₁-C₆ alkyl, and trifluoromethyl; can be reacted with a 3-halopyridine in the presence of a copper catalyst, a ligand, a base, a solvent and optionally an additive. The catalyst can be a copper (I) reagent or a copper (II) reagent. Exemplary catalysts include, but are not limited to, copper (I) chloride (CuCl), copper (II) chloride (CuCl₂), and copper (I) iodide (CuI). In some embodiments, the copper reagent is copper (I) chloride (CuCl). In some embodiments, the reaction can be carried out in the presence of about 0.01 to about 0.4 molar equivalents of copper catalyst compared to the pyrazole starting material. In some embodiments, the reaction can be carried out in the presence of about 0.05 to about 0.25 molar equivalents of copper catalyst compared to the pyrazole starting material. In some embodiments, the reaction can be carried out in the presence of about 0.2 molar equivalents of copper catalyst compared to the pyrazole starting material.

The base in Step (a) can be an inorganic base. Exemplary suitable bases for use in connection with Step (a) include but are not limited sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), sodium phosphate (Na₃PO₄), potassium diphosphate (Na₂HPO₄), potassium phosphate (K₃PO₄), sodium methoxide (NaOCH₃), sodium ethoxide (NaOCH₂CH₃), and the like. In some embodiments, the base is K₃PO₄ or K₂CO₃. In some embodiments, it can be advantageous to use the base in excess compared to the pyrazole starting material. In some embodiments, the base is used in about a 2-fold to about a 5-fold excess. In some embodiments, the base is used in about a 2-fold to about a 3-fold excess. In some embodiments, the base is used in about a 2-fold to excess.

The ligand in the process of Step (a) can be an amine or heteroaryl amine, such as N,N′-dimethylethane-1,2-diamine (DMEDA), triethylenetetreamine (TETA), N,N′-bis(2-hydroxyethyl)ethylenediamine (BHEEA) and 8-hydroxyquinoline. In some embodiments, the reaction can be carried out in the presence of less than an equimolar amount of the ligand to provide further reduction in costs. In some embodiments, the reaction can be carried out in the presence of about 0.08 to about 1.0 molar equivalents of ligand compared to the pyrazole starting material. In some embodiments, the reaction can be carried out in the presence of about 0.4 to about 0.6 molar equivalents of the ligand compared to the pyrazole starting material. In some embodiments, the reaction can be carried out in the presence of about 0.1 to about 0.2 molar equivalents of the ligand compared to the pyrazole starting material.

The process of Step (a) can be conducted in a solvent, such as, acetonitrile (CH₃CN), dioxane, N,N-dimethylformamide (DMF), N-methyl-2-pyrrolidone (NMP), tetrahydrofuran (THF), toluene, and the like. In some embodiments, the solvent is dioxane. In some embodiments, it can be advantageous to carry out the reaction of Step (a) at an elevated temperature. In some embodiments, the reaction is carried out at a temperature between about 50° C. and about 150° C. In some embodiments, the reaction is carried out at a temperature between about 60° C. and about 120° C. In some embodiments, the reaction is carried out at a temperature between about 95° C. and about 115° C.

The process of Step (b) can optionally be carried out when the product of Step (a) is, for example, compound 1B, by acylating the amine to provide a compound of the formula 1B′. In step (b), exemplary acylating agents include acetyl chloride or acetic anhydride. The base in the process of step (b) can be an inorganic base, such as sodium bicarbonate (NaHCO₃). Step (b) can be carried out in the presence of a solvent. In some embodiments, the solvent of step (b) is ethyl acetate (EtOAc) or tetrahydrofuran (THF).

In some embodiments, it can be advantageous to carry out the process of Step (b) at a reduced temperature. In some embodiments, the reaction can be carried out at a temperature of about −10° C. to about 30° C. In some embodiments, the base is used in about a 5% molar excess to about a 5-fold excess. In some embodiments, the base is used in about a 2-fold to about a 3-fold excess. In some embodiments, the base is used in about a 2-fold to excess.

The process of Step (c) can be carried out by a reducing agent, such as borane, sodium borohydride (NaBH₄)/boron trifluoride diethyl etherate (BF₃.Et₂O), sodium bis(2-methoxyethoxy)aluminumhydride (Red-Al), and the like. In some embodiments, it can be advantageous to carry out the reaction of Step (c) in the presence of from about 2.0 to about 5.0 molar equivalents of a reducing agent, such as sodium bis(2-methoxyethoxy)aluminumhydride (Red-Al). In some embodiments, the amount of Red-Al used in the process of Step (c) is about 3.0 molar equivalents. The process of Step (c) can be carried out in the presence of a solvent or a mixture of solvents. In some embodiments, the solvent is tetrahydrofuran (THF), dioxane, diethyl ether (Et₂O), cyclopentylmethylether, or a mixture thereof. In some embodiments, the solvent is a mixture of THF and toluene. It can be advantageous to carry out the reaction of Step (c) at a temperature of from about 0° C. to about 80° C. In some embodiments, it can be advantageous to carry out the process of Step (c) at an elevated temperature. In some embodiments, the temperature of Step (c) can be from about 25° C. to about 50° C.

Alternatively, the processes of the present disclosure can be described according to Scheme 3.

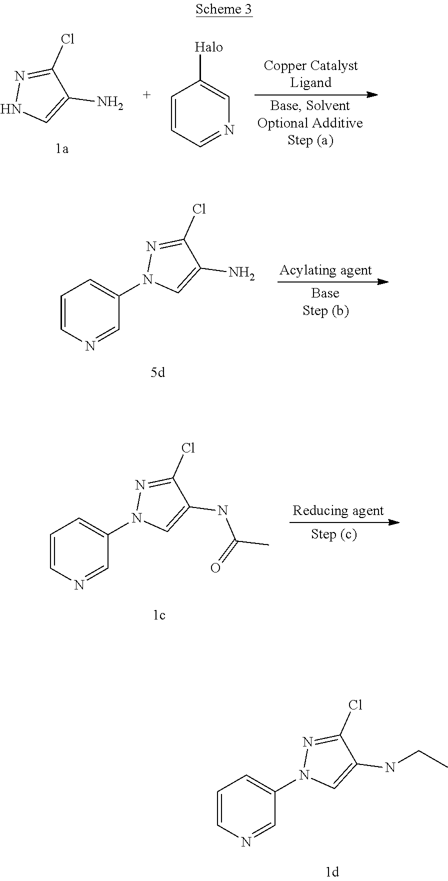

In Step (a) of Scheme 3, the pyrazole starting material 1a, can be reacted with 3-bromopyridine or 3-iodopyridine in the presence of a catalyst, a ligand, a base, and a solvent. The catalyst can be a copper (I) reagent or a copper (II) reagent. Exemplary catalysts include, but are not limited to, copper (I) chloride (CuCl), copper (II) chloride (CuCl₂), and copper (I) iodide (CuI). In some embodiments, the copper reagent is copper (I) chloride (CuCl). In some embodiments, the reaction can be carried out in the presence of about 0.01 to about 0.4 molar equivalents of copper catalyst compared to the pyrazole starting material. In some embodiments, the reaction can be carried out in the presence of about 0.1 to about 0.25 molar equivalents of copper catalyst compared to the pyrazole starting material. In some embodiments, the reaction can be carried out in the presence of about 0.2 molar equivalents of copper catalyst compared to the pyrazole starting material.

The base in Step (a) can be an inorganic base. Exemplary suitable bases for use in connection with Step (a) include but are not limited sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), sodium phosphate (Na₃PO₄), potassium diphosphate (K₂HPO₄), potassium phosphate (K₃PO₄), sodium methoxide (NaOCH₃), sodium ethoxide (NaOCH₂CH₃), and the like. In some embodiments, the base is K₃PO₄ or K₂CO₃. In some embodiments, it can be advantageous to use the base in excess compared to the pyrazole starting material. In some embodiments, the base is used in about a 2-fold to about a 5-fold excess. In some embodiments, the base is used in about a 2-fold to about a 3-fold excess. In some embodiments, the base is used in about a 2-fold to excess.

The ligand in the process of Step (a) can be an amine or heteroaryl amine, such as N,N′-dimethylethane-1,2-diamine (DMEDA), triethylenetetreamine (TETA), N,N′-bis(2-hydroxyethyl)ethylenediamine (BHEEA) and 8-hydroxyquinoline. In some embodiments, the reaction can be carried out in the presence of about 0.08 to about 1.0 molar equivalents of ligand compared to the pyrazole starting material. In some embodiments, the reaction can be carried out in the presence of about 0.4 to about 0.6 molar equivalents of the ligand compared to the pyrazole starting material. In some embodiments, the reaction can be carried out in the presence of about 0.1 to about 0.2 molar equivalents of the ligand compared to the pyrazole starting material.

The process of Step (a) can be conducted in a solvent, such as, acetonitrile (CH₃CN), dioxane, N,N-dimethylformamide (DMF), N-methyl-2-pyrrolidone (NMP), tetrahydrofuran (THF), toluene, and the like. In some embodiments, the solvent is dioxane. In some embodiments, it can be advantageous to carry out the reaction of Step (a) at an elevated temperature. In some embodiments, the reaction is carried out at a temperature between about 50° C. and about 150° C. In some embodiments, the reaction is carried out at a temperature between about 60° C. and about 120° C. In some embodiments, the reaction is carried out at a temperature between about 95° C. and about 115° C.

In step (b), exemplary acylating agents include acetyl chloride or acetic anhydride. The base in the process of step (b) can be an inorganic base, such as sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), potassium phosphate (K₃PO₄), and the like. In some embodiments, the base in step (b) can be sodium bicarbonate (NaHCO₃). Step (b) can be carried out in the presence of a solvent, such as methylene dichloride (DCM), N,N-dimethylformamide (DMF), tetrahydrofuran (THF), ethyl acetate (EtOAc), acetone, acetonitrile (CH₃CN), dimethylsulfoxide (DMSO), and the like. In some embodiments, the solvent of step (b) is ethyl acetate (EtOAc) or tetrahydrofuran (THF). In some embodiments, it can be advantageous to carry out the process of Step (b) at a reduced temperature. In some embodiments, the reaction can be carried out at a temperature of about −10° C. to about 30° C. In some embodiments, the base is used in about a 5% molar excess to about a 5-fold excess. In some embodiments, the base is used in about a 2-fold to about a 3-fold excess. In some embodiments, the base is used in about a 2-fold to excess.

The process of Step (c) can be carried out by a reducing agent, such as borane, sodium borohydride (NaBH₄)/boron trifluoride diethyl etherate (BF₃.Et₂O), sodium bis(2-methoxyethoxy)aluminumhydride (Red-Al), and the like. In some embodiments, it can be advantageous to carry out the reaction of Step (c) in the presence of from about 2.0 to about 5.0 molar equivalents of a reducing agent, such as sodium bis(2-methoxyethoxy)aluminumhydride (Red-Al). In some embodiments, the amount of sodium bis(2-methoxyethoxy)aluminumhydride (Red-Al) used in the process of Step (c) is about 3.0 molar equivalents. The process of Step (c) can be carried out in the presence of a solvent or a mixture of solvents. In some embodiments, the solvent is tetrahydrofuran (THF), dioxane, diethyl ether (Et₂O), cyclopentylmethylether, or a mixture thereof. In some embodiments, the solvent is a mixture of THF and toluene. It can be advantageous to carry out the reaction of Step (c) at a temperature of from about 0° C. to about 80° C. In some embodiments, it can be advantageous to carry out the process of Step (c) at an elevated temperature. In some embodiments, the temperature of Step (c) can be from about 25° C. to about 50° C.

Exemplary methods for the preparation of pesticidal thioethers from compound 1d can be found in, for example, U.S. Pat. No. 9,102,654, incorporated by reference for all it discloses for preparing pesticidal thioethers from a compound of the formula 1d. Exemplary embodiments of such processes can be described as shown in Scheme 4.

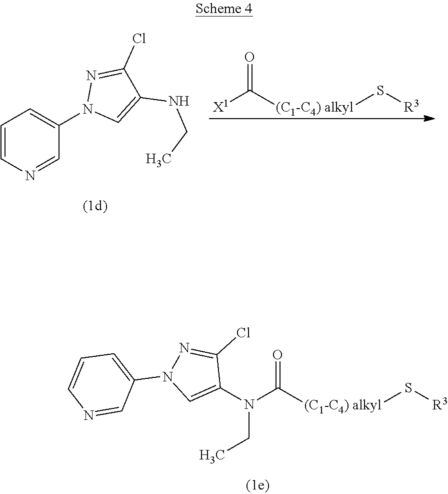

In Scheme 4, 3-chloro-N-ethyl-1-(pyridin-3-yl)-1H-pyrazol-amine (1d) is acylated with activated carbonyl thioethers, indicated as X¹C(═O)(C₁-C₄)-alkyl-S—R³, to produce pesticidal thioethers (1e). In some embodiments, R³ is (C₁-C₄)-haloalkyl, In some embodiments, R³ is CH₂CH₂CF₃.

When X¹ is Cl, the reaction is conducted in a solvent such as ethyl acetate. The reaction may be optionally conducted in the presence of a base such, as sodium bicarbonate, to yield pesticidal thioethers (1e).

When X¹ is OC(═O)(C₁-C₄)-alkyl, the reaction is conducted in the presence of a base preferably sodium bicarbonate, to yield pesticidal thioethers (1e). Alternatively, the reaction may be conducted when X¹ is an activated carboxylic acid, activated by such reagents as 2,4,6-tripropyl-trioxatriphosphinane-2,4,-trioxide (T₃P), carbonyldiimidazole (CDI), dicyclohexylcarbodiimide (DCC) or 1-ethyl-3-(3-dimethyl-aminopropyl)carbodiimide (EDC), preferably 2,4,6-tripropyl-trioxatriphosphinane-2,4,-trioxide and carbonyldiimidazole at temperatures of about 0° C. to about 80° C.; this reaction may also be conducted with uronium or phosphonium activating groups such as O-(7-azabenzotriazol-1-yl)-N,N,N′,N′-tetramethyluronium hexafluorophosphate (HATU) or benzotriazol-1-yl-oxytripyrrolidino-phosphonium hexafluorophosphate (PyBOP), in the presence of an amine base such as diisopropylethylamine or triethylamine, in an aprotic solvent such as N,N-dimethylformamide, tetrahydrofuran, or dichloromethane, at temperatures of about −10° C. to about 30° C., to form pesticidal thioethers (1e).

Activated carbonyl thioethers are prepared from X¹C(═O)(C₁-C₄)-alkyl-S—R³ wherein X¹ is OH, which are prepared by saponifying the corresponding ester thioethers, indicated as X¹C(═O)(C₁-C₄)-alkyl-S—R³, wherein X¹ is O(C₁-C₄)-alkyl, with a metal hydroxide such as lithium hydroxide, in a solvent such as methanol or tetrahydrofuran. Alternatively, X¹C(═O)(C₁-C₄)-alkyl-S—R³, wherein X¹ is OH or O(C₁-C₄)-alkyl may be prepared by the photochemical free-radical coupling of 3-mercaptopropionic acid and esters thereof with 3,3,3-trifluoropropene in the presence of 2,2-dimethoxy-2-phenylacetophenone initiator and long wavelength UV light in an organic solvent. Additionally, X¹C(═O)(C₁-C₄)-alkyl-S—R³, wherein X¹ is OH or O(C₁-C₄)-alkyl may also be prepared by the low temperature free-radical initiated coupling of 3-mercaptopropionic acid and esters thereof with 3,3,3-trifluoropropene in the presence of 2,2′-azobis(4-methoxy-2,4-dimethyl) valeronitrile (V-70) initiator at temperatures of about −50° C. to about 40° C. in a solvent. Preferably, X¹C(═O)(C₁-C₄)-alkyl-S—R³, wherein X¹ is OH or O(C₁-C₄)-alkyl, is prepared by the low temperature free-radical initiated coupling of 3-mercaptopropionic acid and esters thereof with 3,3,3-trifluoropropene in the presence of a two component initiator system of benzoyl peroxide and dimethylaniline or N-phenyldiethanolamine at temperatures of about −50° C. to about 40° C. in a solvent such as toluene or ethyl acetate.

In some embodiments, the present disclosure provides processes for the preparation of pesticidal thioethers.

In some embodiments, the present disclosure provides a process for preparing a compound of the formula 1D

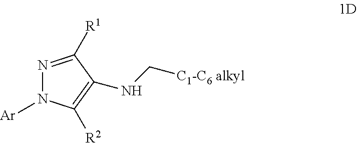

wherein Ar is pyridin-3-yl; and each of R¹ and R² is independently selected from the group consisting of H, F, Cl, Br, I, C₁-C₆ alkyl and trifluoromethyl; comprising

a. Contacting a Compound of the Formula 1A

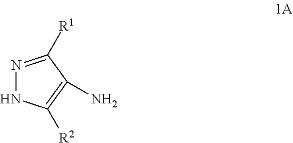

wherein each of R¹ and R² is independently selected from the group consisting of H, F, Cl, Br, I, C₁-C₆ alkyl and trifluoromethyl; with a 3-halopyridine in the presence of a catalyst, a ligand, a base, and a solvent, to provide a compound of the formula 1B

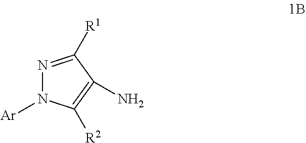

wherein Ar is pyridin-3-yl; and each of R¹ and R² is independently selected from the group consisting of H, F, Cl, Br, I, C₁-C₆ alkyl and trifluoromethyl; or

b. Contacting a Compound of the Formula 1B

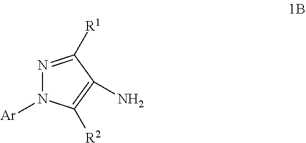

wherein Ar is pyridin-3-yl; and each of R¹ and R² is independently selected from the group consisting of H, F, Cl, Br, I, C₁-C₆ alkyl and trifluoromethyl; with an acylating agent to provide a compound of the formula 1B′

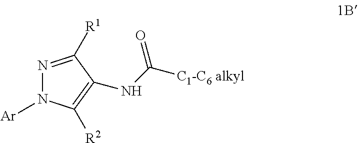

wherein Ar is pyridin-3-yl; and each of R¹ and R² is independently selected from the group consisting of H, F, Cl, Br, I, C₁-C₆ alkyl and trifluoromethyl; or

c. Contacting a Compound of the Formula 1B′

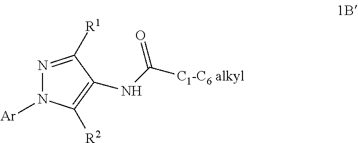

wherein Ar is pyridin-3-yl; and each of R¹ and R² is independently selected from the group consisting of H, F, Cl, Br, I, C₁-C₆ alkyl and trifluoromethyl; with a reducing agent to provide a compound of the formula 1D.

In some embodiments, the present disclosure provides a process for preparing a compound of the formula 1d

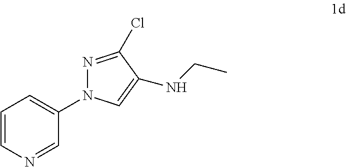

comprising

a. Contacting a Compound of the Formula 1a

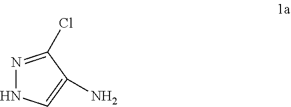

with a 3-halopyridine in the presence of a catalyst, a ligand, a base and a solvent to provide a compound of the formula 5d

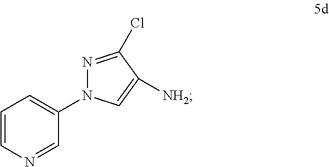 [SMILES: Nc1cn(-c2cccnc2)nc1Cl]

b. Contacting a Compound of the Formula 5d

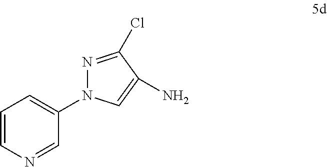 [SMILES: Nc1cn(-c2cccnc2)nc1Cl]

with an acylating agent in the presence of a base and a solvent to provide a compound of the formula 1c

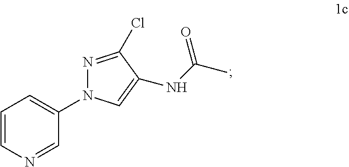 [SMILES: CC(=O)Nc1cn(-c2cccnc2)nc1Cl]

or

c. Contacting a Compound of the Formula 1c

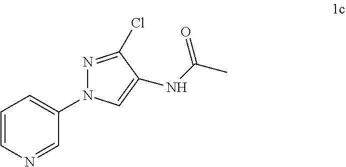 [SMILES: CC(=O)Nc1cn(-c2cccnc2)nc1Cl]

with a reducing agent in the presence of a solvent to provide the compound of the formula 1d.

In some embodiments, the processes described here in comprise Step (a) and Step (b). In some embodiments, the processes described here in comprise Step (a) and Step (c). In some embodiments, the processes described here in comprise Step (a), Step (b) and Step (c). In some embodiments, the processes of the present disclosure can be carried out in connection with processes for preparing pesticidal thioethers, such as those described in U.S. Pat. No. 9,102,654.

### EXAMPLES

These examples are for illustration purposes and are not to be construed as limiting this disclosure to only the embodiments disclosed in these examples.

Starting materials, reagents, and solvents that were obtained from commercial sources were used without further purification. Melting points are uncorrected. Examples using “room temperature” were conducted in climate controlled laboratories with temperatures ranging from about 20° C. to about 24° C. Molecules are given their known names, named according to naming programs within Accelrys Draw, ChemDraw, or ACD Name Pro. If such programs are unable to name a molecule, such molecule is named using conventional naming rules. ¹H NMR spectral data are in ppm (δ) and were recorded at 300, 400, 500, or 600 MHz; ¹³C NMR spectral data are in ppm (δ) and were recorded at 75, 100, or 150 MHz, and ¹⁹F NMR spectral data are in ppm (δ) and were recorded at 376 MHz, unless otherwise stated.

3-Chloro-1H-pyrazol-4-amine hydrochloride, compound 1a, was prepared according to the method described in U.S. Pat. No. 9,102,655, incorporated herein by reference for the preparation of compound Ia, referred to therein as compound 1a.

### COMPOUND EXAMPLES

### Example 1—Preparation of 3-chloro-1(pyridin-3-yl)-1H-pyrazol-4-amine (5d)

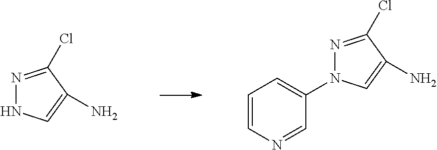 [SMILES: Nc1c[nH]nc1Cl ; Nc1cn(-c2cccnc2)nc1Cl]

A three-neck round bottomed flask (100 mL) was charged with copper (I) chloride (0.627 g, 6.33 mmol), N,N′-dimethylethane-1,2-diamine (1.12 g, 12.7 mmol), 3-chloro-1H-pyrazol-4-amine hydrochloride (5.85 g, 38.0 mmol), potassium carbonate (8.75 g, 63.3 mmol), and dioxane (50 mL), and the mixture was stirred under nitrogen for 10 min. 3-Bromopyridine (3.05 mL, 31.6 mmol) was added, and the mixture was heated at 80° C. for 18 h. The reaction was allowed to cool to 20° C. and filtered through a Celite® pad. The pad was rinsed with ethyl acetate (2×20 mL) and the combined filtrates were concentrated. Purification by flash column chromatography using 0-80% ethyl acetate/hexanes as eluent provided the title compound as a light yellow solid (3.56 g, 58%): ¹H NMR (400 MHz, DMSO-d₆) δ 8.95 (dd, J=2.6, 0.8 Hz, 1H), 8.45 (dd, J=4.7, 1.4 Hz, 1H), 1H), 8.07 (ddd, J=8.4, 2.4, 1.4 Hz, 1H), 7.90 (s, 1H), 7.48 (ddd, J=8.3, 4.7, 0.8 Hz, 1H), 4.42 (s, 2H); ¹³C NMR (101 MHz, DMSO-d₆) δ 146.35, 138.53, 135.72, 132.09, 130.09, 124.29, 124.11, 114.09; ESIMS m/z 195 ([M+H])+).

### Example 2—Preparation of N-(3-Chloro-1-(pyridin-3-yl)-1H-pyrazol-4-yl)acetamide (1c)

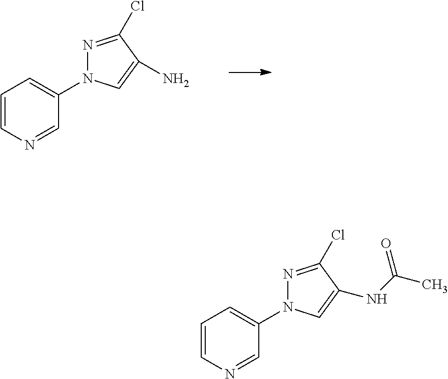 [SMILES: Nc1cn(-c2cccnc2)nc1Cl ; CC(=O)Nc1cn(-c2cccnc2)nc1Cl]

A three-neck round bottomed flask (100 mL) was charged with 3-chloro-1(pyridin-3-yl)-1H-pyrazol-4-amine (1.00 g, 5.14 mmol) and ethyl acetate (10 mL). Sodium bicarbonate (1.08 g, 12.9 mmol) was added, followed by dropwise addition of acetic anhydride (0.629 g, 6.17 mmol) at <20° C. The reaction was stirred at 20° C. for 2 h to afford a suspension, at which point thin layer chromatography analysis [TLC, eluent: ethyl acetate] indicated that the reaction was complete. The reaction was diluted with water (50 mL) and the resulting suspension was filtered. The solid was rinsed with water (10 mL) followed by methanol (5 mL). The solid was further dried under vacuum at 20° C. to afford the desired product as a white solid (0.804 g, 66%): mp 169-172° C.; ¹H NMR (400 MHz, DMSO-d₆) δ 9.84 (s, 1H), 9.05 (dd, J=2.8, 0.8 Hz, 1H), 8.82 (s, 1H), 8.54 (dd, J=4.7, 1.4 Hz, 1H), 8.20 (ddd, J=8.4, 2.8, 1.4 Hz, 1H), 7.54, (ddd, J=8.3, 4.7, 0.8 Hz, 1H), 2.11 (s, 3H); ¹³C NMR (101 MHz, DMSO-d₆) δ 168.12, 147.46, 139.42, 135.46, 133.60, 125.47, 124.21, 122.21, 120.16, 22.62; EIMS m/z 236 ([M]⁺).

### Example 3—Preparation of 3-chloro-N-ethyl-1-(pyridin-3-yl)-1H-pyrazol-amine

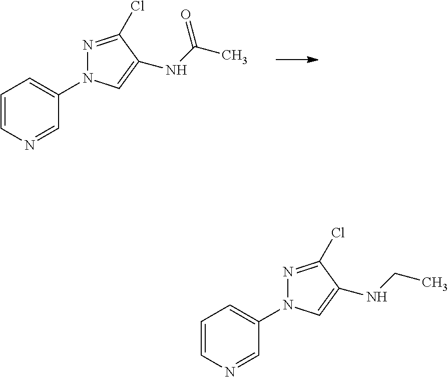 [SMILES: CC(=O)Nc1cn(-c2cccnc2)nc1Cl ; CCNc1cn(-c2cccnc2)nc1Cl]

To a 25 mL round bottom flask was added N-(3-chloro-1-(pyridin-3-yl)-1H-pyrazol-4-yl)-N-ethylacetamide (1.0 g, 4.2 mmol), and anhydrous THF (6.0 mL) leading to a white suspension. The suspension was cooled in an ice-water bath to 6° C. Sodium bis(2-methoxyethoxy)aluminum dihydride (Red-Al, 60 wt % in toluene, 3.52 mL, 2.5 eq.) was added slowly with via syringe over 10 min while keeping pot temp <10° C. The thick suspension gradually turned clear yellow solution during Red-Al addition. The reaction mixture was slowly warmed up to 25° C. over 1.5 h. LC indicated 83.8% conversion. The solution was heated to 50° C. and stirred for 4.5 h. LC indicated 88.1% conversion. The solution was cooled down to 20° C. NaOH (2 M in H₂O, 5 mL) was added to quench the reaction leading to white suspension. Water (20 mL) was added and the mixture separated into two phases. The aqueous phase was separated and extracted with EtOAc (2×20 mL). The organic layers were combined, dried over anhydrous sodium sulfate, and concentrated by rotavap to afford a crude product as a red-brown oil (0.758 g). LC assay using di-n-propyl phthalate as internal standard indicated 78 wt %, 0.591 g active, 62.8% yield. ESIMS m/z 222 ([M+H]⁺).

### Example 4—Alternative preparation of 3-chloro-N-ethyl-1-(pyridin-3-yl)-1H-pyrazol-amine

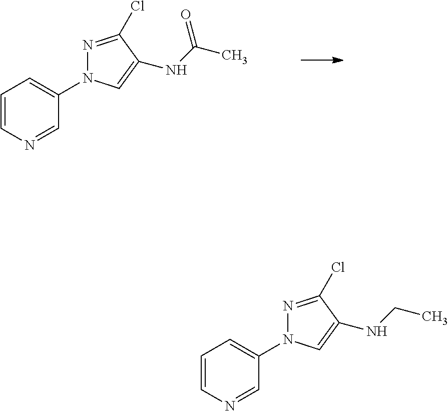 [SMILES: CC(=O)Nc1cn(-c2cccnc2)nc1Cl ; CCNc1cn(-c2cccnc2)nc1Cl]

To a 25 mL round bottom flask was added N-(3-chloro-1-(pyridin-3-yl)-1H-pyrazol-4-yl)-N-ethylacetamide (1.0 g, 4.2 mmol) and anhydrous THF (9 mL) leading to a while suspension. The flask was cooled in an ice-water bath to 5° C. Red-Al (60 wt % in toluene, 4.3 mL, 3.0 eq.) was added slowly via syringe over 15 min while keeping pot temp <10° C. The thick suspension gradually turned clear yellow solution during the addition of the Red-Al. The reaction mixture was slowly warmed up to 25° C. over 3 h, then was stirred at 25° C. for 17 h. NaOH (2 M in H₂O, 6 mL) was added to quench the reaction leading to a thick slurry. Water (20 mL) was added and the two layers were separated. The bottom aqueous phase was extracted with Et₂O (3×20 mL). Combined organic layers were dried over anhydrous sodium sulfate and concentrated by rotavap to afford crude product as red-brown oil 0.91 g. LC assay using di-n-propyl phthalate as the internal standard indicated 61 wt %, 0.555 g active, 59.0% yield. ESIMS m/z 222 ([M+H]⁺).

### Alternative preparation of 3-Chloro-N-ethyl-1-(pyridin-3-yl)-1H-pyrazol-4-amine

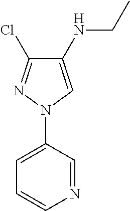

A 100 mL 3-neck round bottom flask was charged with N-(3-chloro-1-(pyridin-3-yl)-1H-pyrazol-4-yl)acetamide (475 mg, 2.01 mmol) and tetrahydrofuran (10 mL). Borontrifluoride etherate (0.63 mL, 5.02 mmol) was added and the mixture was stirred for 15 min to give a suspension. Sodium borohydride (228 mg, 6.02 mmol) was added and the reaction was heated at 60° C. for 4 h, at which point thin layer chromatography analysis [Eluent: ethyl acetate, sample was prepared by treatment of reaction mixture with hydrochloric acid, followed by sodium bicarbonate basification and ethyl acetate extraction] indicated that the reaction was complete. Water (10 mL) and concentrated hydrochloric acid (1 mL) were added and the reaction was heated at 60° C. for 1 h. The reaction mixture was cooled to room temperature and distilled to remove tetrahydrofuran. The mixture was neutralized with saturated aqueous sodium bicarbonate to pH ˜8 to afford a suspension, which was stirred for 1 h and filtered. The filter cake was rinsed with water (10 mL) and dried under vacuum to afford a white solid (352 mg, 79%): mp 93-96° C.; ¹H NMR (400 MHz, DMSO-d₆) δ 8.99 (d, J=2.7 Hz, 1H), 8.44 (dd, J=4.6, 1.4 Hz, 1H), 8.10 (ddd, J=8.4, 2.7, 1.4 Hz, 1H), 8.06 (s, 1H), 7.50 (dd, J=8.4, 4.7 Hz, 1H), 4.63 (t, J=6.0 Hz, 1H), 3.06-2.92 (m, 2H), 1.18 (t, J=7.1 Hz, 3H); ¹³C NMR (101 MHz, DMSO-d₆) δ 146.17, 138.31, 135.81, 132.82, 130.84, 124.10, 123.96, 112.23, 40.51, 14.28; EIMS m/z 222 ([M]⁺).

### Example 5—Preparation of N-(3-Chloro-1-(pyridin-3-yl)-1H-pyrazol-4-yl)-N-ethyl-3-((3,3,3-trifluoropropyl)thio)propanamide (Compound 6.9)

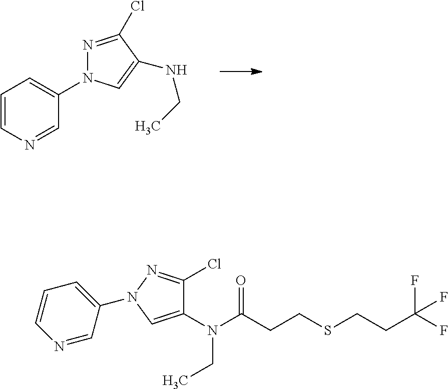 [SMILES: CCNc1cn(-c2cccnc2)nc1Cl ; CCN(C(=O)CCSCCC(F)(F)F)c1cn(-c2cccnc2)nc1Cl]

A three-neck round bottomed flask (100 mL) was charged with 3-chloro-N-ethyl-1-(pyridin-3-yl)-1H-pyrazol-amine (5.00 g, 22.5 mmol) and ethyl acetate (50 mL). Sodium bicarbonate (4.72 g, 56.1 mmol) was added, followed by dropwise addition of 3-((3,3,3-trifluoropropyl)thio)propanoyl chloride (5.95 g, 26.9 mmol) at <20° C. for 2 h, at which point HPLC analysis indicated that the reaction was complete. The reaction was diluted with water (50 mL) (off-gassing) and the layers separated. The aqueous layer was extracted with ethyl acetate (20 mL) and the combined organic layers were concentrated to dryness to afford a light brown solid (10.1 g, quantitative). A small sample of crude product was purified by flash column chromatography using ethyl acetate as eluent to obtain an analytical reference sample: mp 79-81° C.; ¹H NMR (400 MHz, DMSO-d₆) δ 9.11 (d, J=2.7 Hz, 1H), 8.97 (s, 1H), 8.60 (dd, J=4.8, 1.4 Hz, 1H), 8.24 (ddd, J=8.4, 2.8, 1.4 Hz, 1H), 7.60 (ddd, J=8.4, 4.7, 0.8 Hz, 1H), 3.62 (q, J=7.2 Hz, 2H), 2.75 (t, J=7.0 Hz, 2H), 2.66-2.57 (m, 2H), 2.57-2.44 (m, 2H), 2.41 (t, J=7.0 Hz, 2H), 1.08 (t, J=7.1 Hz, 3H); ESIMS m/z 407 ([M+H]⁺).

What is claimed is:

1. A process for preparing a compound of the formula 1D 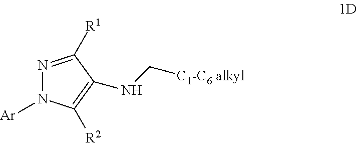
wherein Ar is pyridin-3-yl; and each of R¹ and R² is independently selected from the group consisting of H, F, Cl, Br, I, C₁-C₆ alkyl and trifluoromethyl;
comprising
(a) contacting a compound of the formula 1A 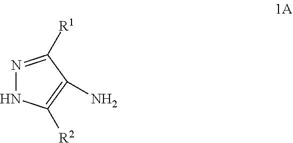
wherein each of R¹ and R² is independently selected from the group consisting of H, F, Cl, Br, I, C₁-C₆ alkyl, and trifluoromethyl; with a 3-halopyridine in the presence of a catalyst, a ligand, a base and a solvent to provide a compound of the formula 1B 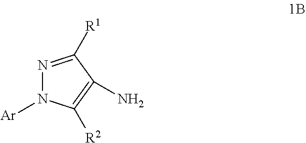
wherein Ar is pyridin-3-yl; and each of R¹ and R² is independently selected from the group consisting of H, F, Cl, Br, I, C₁-C₆ alkyl and trifluoromethyl;
(b) contacting a compound of the formula 1B 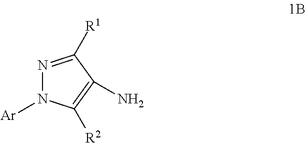
wherein Ar is pyridin-3-yl; and each of R¹ and R² is independently selected from the group consisting of H, F, Cl, Br, I, C₁-C₆ alkyl and trifluoromethyl; with an acylating agent in the presence of a base and a solvent to provide a compound of the formula 1B′ 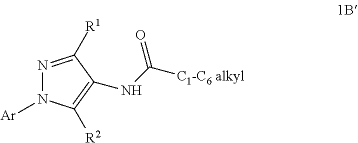
wherein Ar is pyridin-3-yl; and each of R¹ and R² is independently selected from the group consisting of H, F, Cl, Br, I, C₁-C₆ alkyl and trifluoromethyl; and
(c) contacting a compound of the formula 1B′ 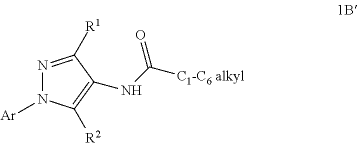
wherein Ar is pyridin-3-yl; and each of R¹ and R² is independently selected from the group consisting of H, F, Cl, Br, I, C₁-C₆ alkyl and trifluoromethyl; with a reducing agent in the presence of a solvent to provide a compound of the formula 1D.

2. The process of claim 1, wherein the catalyst in step (a) is copper (I) chloride (CuCl), copper (II) chloride (CuCl₂), copper (I) bromide (CuBr), or copper (I) iodide (CuI).

3. The process of claim 1, wherein the ligand in step (a) is selected from the group consisting of N,N′-dimethylethane-1,2-diamine (DMEDA), triethylenetetreamine (TETA), N,N′-bis(2-hydroxyethyl)ethylenediamine (BHEEA) and 8-hydroxyquinoline.

4. The process of claim 1, wherein the base in step (a) is selected from the group consisting of sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), sodium phosphate (Na₃PO₄), potassium diphosphate (Na₂HPO₄), potassium phosphate (K₃PO₄), sodium methoxide (NaOCH₃) and sodium ethoxide (NaOCH₂CH₃).

5. The process of claim 4, wherein the base in step (a) is K₂CO₃.

6. The process of claim 1, wherein the solvent in step (a) is acetonitrile (CH₃CN), dioxane, N,N-dimethylformamide (DMF), N-methyl-2-pyrrolidone (NMP), tetrahydrofuran (THF), toluene, or dimethylsulfoxide (DMSO).

7. The process of claim 1, wherein the acylating agent in step (b) is acetyl chloride or acetic anhydride.

8. The process of claim 1, wherein the base in step (b) is selected from the group consisting of sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄) and potassium phosphate (K₃PO₄).

9. The process of claim 8, wherein the base in step (b) is sodium bicarbonate (NaHCO₃).

10. The process of claim 1, wherein the solvent of step (b) is selected from the group consisting of methylene dichloride (DCM), N,N-dimethylformamide (DMF), tetrahydrofuran (THF), ethyl acetate (EtOAc), acetone, acetonitrile (CH₃CN) and dimethylsulfoxide (DMSO).

11. The process of claim 10, wherein the solvent is ethyl acetate (EtOAc) or tetrahydrofuran (THF).

12. The process of claim 1, wherein the reducing agent in step (c) is selected from the group consisting of Red-Al, borane, and sodium borohydride (NaBH₄)/boron trifluoride diethyl etherate (BF₃.Et₂O).

13. The process of claim 1, wherein the solvent of step (c) is tetrahydrofuran (THF), dioxane, diethyl ether (Et₂O), cyclopentylmethylether, or a mixture thereof.

14. The process of claim 13, wherein the solvent is THF.

15. The process of claim 1, wherein the 3-halopyridine is 3-bromopyridine.

16. The process of claim 1, wherein R² is H.

17. The process of claim 1, wherein R¹ is Cl.

18. A process comprising
(a) contacting a compound of the formula 1A 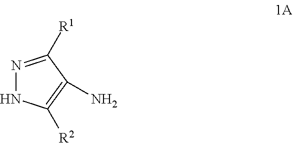
wherein each of R¹ and R² is independently selected from the group consisting of H, F, Cl, Br, I, C₁-C₆ alkyl, and trifluoromethyl; with a 3-halopyridine in the presence of a catalyst, a ligand, a base and a solvent to provide a compound of the formula 1B 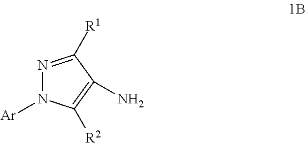
wherein Ar is pyridin-3-yl; and each of R¹ and R² is independently selected from the group consisting of H, F, Cl, Br, I, C₁-C₆ alkyl and trifluoromethyl.

19. A process comprising
(b) contacting a compound of the formula 1B 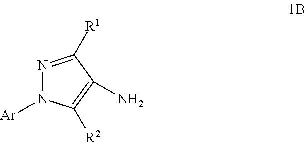
wherein Ar is pyridin-3-yl; and each of R¹ and R² is independently selected from the group consisting of H, F, Cl, Br, I, C₁-C₆ alkyl and trifluoromethyl; with an acylating agent in the presence of a base and a solvent to provide a compound of the formula 1B′ 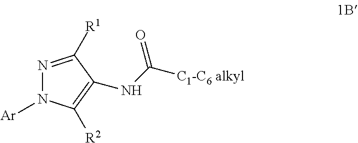
wherein Ar is pyridin-3-yl; and each of R¹ and R² is independently selected from the group consisting of H, F, Cl, Br, I, C₁-C₆ alkyl and trifluoromethyl.

20. A process comprising
(c) contacting a compound of the formula 1B′ 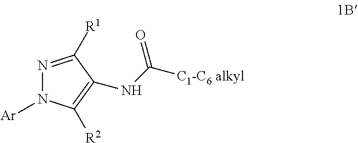
wherein Ar is pyridin-3-yl; and each of R¹ and R² is independently selected from the group consisting of H, F, Cl, Br, I, C₁-C₆ alkyl and trifluoromethyl; with a reducing agent in the presence of a solvent to provide a compound of the formula 1D 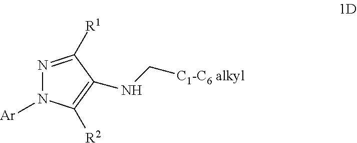
wherein Ar is pyridin-3-yl; and each of R¹ and R² is independently selected from the group consisting of H, F, Cl, Br, I, C₁-C₆ alkyl and trifluoromethyl.
